(19) United States
(12) Patent Application Publication
(10) Pub. No.: US 2004/0055095 A1
(43) Pub. Date: Mar. 25, 2004

(54) Oxidizing compositions comprising a phosphonic acid type chelant and a conditioning agent and methods of treating hair

(75) Inventors: Graham Neil McKelvey, Surrey (GB); James Charles Dunbar, Morrow, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/667,960

(22) Filed: Sep. 22, 2003

Related U.S. Application Data
(63) Continuation of application No. PCT/US02/08478, filed on Mar. 19, 2002.

(30) Foreign Application Priority Data
Mar. 20, 2001 (GB) 0106946.7
Nov. 30, 2001 (GB) 0128751.5

(51) Int. Cl. A61K 7/13

(52) U.S. Cl. 8/405; 8/421

(57) ABSTRACT

Compositions suitable for treating hair comprising an oxidizing agent, a conditioning agent selected from silicone materials, especially nonvolatile silicone and amino functionalised silicones, cationic surfactants, cationic polymers, alkoxylated amines amines and mixtures thereof and a chelant selected from phosphonic acid chelants, salts thereof, derivatives thereof and mixtures thereof and methods of treating hair. The compositions of the invention are useful for depositing high amount of conditioning agent and reducing oxidative hair damage during oxidative treatment of hair such as oxidative hair dyeing and hair bleaching.

### CROSS REFERENCE TO RELATED APPLICATION

The application is a continuation of International application PCT/US02/08478 (Case CM2631M) filed on Mar. 19, 2002.

### FIELD

The present invention relates to oxidizing compositions comprising a phosphonic acid type chelant and a conditioning agent and to methods of treating hair. The compositions and methods disclosed provide enhanced deposition of conditioning agents on hair subjected to oxidative treatments such as bleaching, oxidative hair dyeing and perming.

### BACKGROUND

Melanin is a natural pigment found in hair. Melanin and hair-forming cells are naturally produced in the hair bulb at the root of the hair. As new cells are produced, the older ones are pushed upwards out of the skin to form the hair shaft, which is the part of the hair that can be seen above the scalp. Hair can be schematically described as being made of a center part called the cortex, which contains the melanin, and an outer layer called the cuticle. It is the cortex that gives hair its special qualities such as elasticity and curl.

The hair shaft is made of dead cells that have turned into a mixture of different forms of the special hair protein, keratin. Keratin contains high concentrations of a particular amino acid called cystine. Every cystine unit contains two cysteine amino acids in different chains, which have come to lie near each other and are linked together by two sulphur atoms, forming a very strong chemical bond known as a disulphide linkage. This cross-linking by disulphide linkages between the keratin chains accounts for much of the strength of the hair.

Bleaching and dyeing (or coloring) of hair has become increasingly popular over the past years. Younger people may want to change the natural color of their hair to a more fashionable one, while older people may also use dyeing compositions to conceal gray hair. As people grow older, the production of melanin slows, giving more and more gray hair over time.

Melanin can also be purposely altered by chemical treatments to give lighter shades. The lightening is achieved by oxidizing the melanin pigments with an oxidizing composition, usually in alkaline solution. The oxidizing compositions (bleaches) comprise an oxidizing agent, usually hydrogen peroxide. Other suitable oxidizing agents include potassium, sodium and ammonium salts of perborate, percarbonate, persulfate and percarbamide.

Bleaches are also used during oxidative dyeing treatments. Oxidative (or “permanent”) dye compositions comprise “precursor dyes” which are small molecules capable of diffusing into the hair. These molecules mainly belong to three classes of aromatic compounds: diamines, aminophenols and phenols. They are sufficiently small to diffuse in the hair shaft where, once activated by an oxidizing agent such as hydrogen peroxide, they further react with other precursors to form larger colored complexes. Oxidative hair dye compositions commonly contain, in addition to the dye precursors and a source of peroxide, a variety of additional cosmetic and peroxide stabilizing agents.

Oxidizing agents can activate oxidative dye precursors across a range of pH. However, it is known that enhanced dye oxidation can be achieved via the use of a hair-swelling agent (HSA) that can adjust the pH of the oxidizing solution. Such HSA's further enhance the oxidizing and dyeing process by swelling the hair fibers to aid both the diffusion of the peroxide and dyeing agents into the hair and enabling faster, more thorough dye oxidization and hair dyeing. Preferred hair-swelling agents for adjusting the pH of peroxide hair oxidizing compositions are aqueous alkaline solutions containing ammonia (ammonium hydroxide) or monoethanolamine (MEA).

Low levels of chelants are routinely used as stabilizers or preservatives in various oxidizing compositions. For example, EDTA (ethylenediaminetetraacetic acid) is commonly used as a stabilizer in hydrogen peroxide solution, which would otherwise decompose too rapidly and could not be stored for a long time. Amounts as low as 0.1% by weight of the oxidizing composition are usually used to stabilize the oxidizing agent contained in said oxidizing compositions.

Oxidative treatments of hair such as bleaching (decoloration) and oxidative dyeing give good results and are very commonly used. They are however not without drawbacks. The oxidizing agents used for bleaching and oxidative dyeing damage hair to some extent. The mechanism by which damage is caused to the hair fibers is not perfectly understood. However, it is known that some of the disulphide bonds linking the keratin chains break in the presence of oxidizing compositions. Repeated oxidative treatments leave weak, brittle hairs, which have little shine and luster. An enormous effort has been made to address this problem over the past years, and various solutions have been proposed.

U.S. Pat. No. 4,138,478 discloses agents for reducing the damage to hair during bleaching and dyeing by the use of a water-soluble 3-amino-1-hydroxypropane-1,1-diphosphonic compound for protecting hair from damage by “nascent oxygen”. According to this patent, “the diphosphonic compound is substantively adsorbed by the hair and acts to hinder degradation of the hair by nascent oxygen which is either present therewith or which is substantially added”. Other protective compounds such as hydroxyethane-1,1diphosphonic acid (HEDP) and ethylenediaminetetramethylene phosphonic acid (EDTMP) are disclosed at low levels in U.S. Pat. Nos. 3,202,579 and 3,542,918.

Today, most dyeing or bleaching compositions are sold with a conditioner that is applied on hair after the bleaching or dyeing composition has been rinsed off. Examples of conditioning agents are silicones, cationic surfactants and cationic polymers. Conditioners do not bring the hair back to its initial condition but conceal the damage under a protective layer of the conditioning agent, which results in an improved feel of the hair.

It has now been surprisingly found that chelants having a phosphonic acid moiety substantially increase the deposition of conditioning agents (or “conditioners”) on hair during or after an oxidative treatment such as bleaching or oxidative dyeing resulting in longer-lasting improved hair feel. It has been found that for this purpose the chelants can be applied to hair either at the same time as the oxidizing composition or as a pre-treatment. Chelants, conditioning agents and oxidizing compositions are preferably applied simultaneously.

Without being bound by theory, it is believed that the mechanism of deposition of the conditioning agents is twofold. Firstly, phosphonic acid type chelants are believed to trap very efficiently environmental and intrinsic heavy metal ions such as iron, manganese and copper. It is believed that in the absence of chelants, these heavy metal ions react with hydrogen peroxide and form free radicals that weaken the structure of hair by destroying the disulphide bonds of keratin. It is furthermore believed that non-cationic conditioners such as silicones deposit less efficiently on damaged hair and therefore chelants, by reducing oxidative hair damage, substantively increase the efficiency of the deposition of the conditioners.

Secondly, in 1888 Hofmeister classified ions by their ability to enhance or decrease the solubility of proteins in water (known as the Hofmeister or lyotropic series). Phosphates were found to be particularly good at precipitating proteins and, although not wishing to be bound by theory, the Applicant believes that phosphonate chelants interact with the conditioning agents in a similar way when used in the same composition, resulting in enhanced deposition.

### SUMMARY

The present invention is directed to a composition suitable for treating hair comprising, by weight of the composition:

a) an oxidizing agent;

b) a conditioning agent selected from silicone materials, especially nonvolatile silicone and amino functionalised silicones, cationic surfactants, cationic polymers, alkoxylated amines and mixtures thereof; and

c) a chelant selected from phosphonic acid type chelant, salts thereof, derivatives thereof and mixtures thereof.

The present invention is also directed to a method of treating hair comprising the step of contacting hair with a composition according to the above definition.

The present invention is also directed to a method of treating hair comprising the subsequent steps of:

i) contacting hair with a first composition comprising:

a) a conditioning agent selected from silicone materials, especially nonvolatile silicone and amino functionalised silicones, cationic polymers, alkoxylated amines and mixtures thereof and;

b) a cheiant selected from phosphonic acid type chelants, salts thereof, derivatives thereof and mixtures thereof;

ii) optionally rinsed said first composition off hair;

iii) contacting hair with a second composition comprising an oxidizing agent immediately after step i) or ii).

The present invention is also directed to a method of treating hair comprising the steps of:

i) contacting hair with a first composition comprising an oxidizing agent and a chelant selected from phosphonic acid type chelants, salts thereof, derivatives thereof and mixtures thereof;

ii) optionally rinsing said first composition off hair; and

iii) contacting hair immediately after step i) or ii) with a second composition comprising a conditioning agent selected from silicone materials, especially nonvolatile silicone and amino functionalised silicones, cationic surfactants, cationic polymers, alkoxylated amines and mixtures thereof.

The present invention is further directed to kits adapted for treating hair according to these methods.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

As used herein the term “hair” to be treated may be “living” i.e. on a living body or may be “non-living” i.e. in a wig, hairpiece or other aggregation of non-living keratinous fibers. Mammalian, preferably human hair is preferred. However wool, fur and other keratin containing fibers are suitable substrates for the compositions according to the present invention.

As used herein, the term “oxidizing composition” means a composition comprising at least one oxidizing agent suitable for use on hair, such as hydrogen peroxide, sodium, potassium, ammonium or other salts of perborate, percarbonate, persulfate and percarbamide. Non-limiting examples of such compositions are oxidative dye compositions and bleaching compositions.

As used herein the term “oxidative treatment of hair” or “hair treatment comprising at least one oxidative step” is used in the broad sense in that it is intended to encompass all treatments of hair comprising at least one step of contacting hair with at least one oxidizing composition. Preferred examples of oxidative treatment for human hair are bleaching, dyeing or perming.

As used herein the term “immediately” means within about 1 hour, preferably within about 30 mn, more preferably within about 15 mn.

All percentages are by weight of the total composition unless specifically stated otherwise. When more than one composition are used during a treatment, the total weight to be considered is the total weight of all the compositions applied on hair simultaneously (i.e. the weight found “on head”) unless otherwise specified. All ratios are weight ratios unless specifically stated otherwise.

All cited references are incorporated herein by reference in their entireties. Citation of any reference is not an admission regarding any determination as to its availability as prior art to the claimed invention.

Chelants

The term “chelant” (or “chelating agent” or “sequestering agent”) is well known in the art and refers to a molecule or a mixture of different molecules each capable of forming a chelate with a metal ion. A chelate is an inorganic complex in which a compound (chelant) is coordinated to a metal ion at two or more points so that there is a ring of atoms including the metals. Chelants contain two or more electron donor atoms that form the coordination bonds with the metal ion.

Chelants are well known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference.

When related to chelants, the terms “salts and derivatives thereof” mean all salts and derivatives comprising the same functional structure (same chemical backbone) as the chelant they are referring to and having similar or better chelating properties. These terms include alkali metal, alkaline earth, ammonium, substituted ammonium salts (e.g monoethanolammonium, diethanolammonium, triethanolammonium), esters of chelants having an acidic moeity and mixtures thereof, in particular all sodium, potassium or ammonium salts. The term “Derivatives” also includes “chelating surfactant” compounds (these are chelants modified to bear a surfactant moiety while keeping the same chelating functionality, see U.S. Pat. No. 5284972, “N-acyl-N,N′,N′-ethylenediaminetriacetic acid” for an example of modified ethylenediaminetriacetic acid). The term “Derivatives” also includes large molecules comprising one or more chelating groups having the same functional structure as the parent chelant. Examples of these large molecules include polymeric EDDS (ethylenediaminedisuccinic acid) made of unit block according to the following structure:

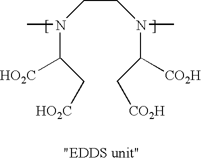

and disclosed in U.S. Pat. No. 5,747,440 Kellett et al. “Polymeric” phosphonic acid type chelant derivatives based on this model and repeating the functional structure of the phosphonic acid type chelants described hereinafter can be synthesized using the teaching of this document.

The compositions according to the present invention comprise a chelant selected from phosphonic acid type chelants, salts thereof, derivatives thereof and mixtures thereof. Phosphonic acid type chelants are defined as chelants comprising a phosphonic acid moiety (—PO₃H₂) or its derivative —PO₃R₂ wherein R is an alkyl or aryl radical.

Suitable phosphonic acid type chelants include aminopolyphosphonic acids and polyphosphonic acids.

Suitable polyphosphonic acids and polyphosphonic derivatives include compounds having the formula (I) below:

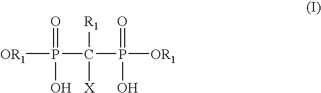

wherein: X is —OH or —NH_{2;}

R₁ is an aryl radical, an aliphatic radical having 1 to 5 carbon atoms or the radical (Ia) below:

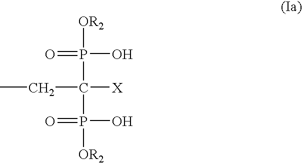

and wherein each R₂ are independently selected from H or alkyl radicals having from 1 to 5 carbon atoms.

Suitable aminopolyphosphosphonic acid and aminopolyphosphosphonic derivatives include chelants having the following formula (II):

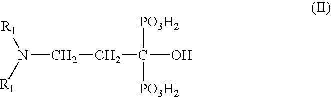

wherein each R₁ are independently selected from H or C₁-C₃ alkyl.

Preferred phosphonic acid type chelants for use herein have the formula (III) below:

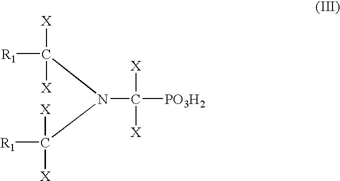

wherein each X are independently selected from hydrogen or alkyl radicals, preferably hydrogen or alkyl radicals having from 1 to 4 carbon atoms, preferably hydrogen; and each R₁ are independently selected from —PO₃H₂ or a group having the formula (IIIa) below:

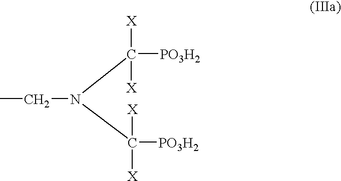

Preferred chelants according to Formula (III) for use herein are aminotri-(1-ethylphosphonic acid), ethylenediaminetetra-(1-ethylphosphonic acid), aminotri-(1-propylphosphonic acid), aminotri-(isopropylphosphonic acid) and chelants having the formula (IV) below:

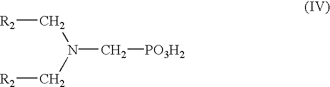

wherein each R₂ are independently selected from —PO₃H₂ or a group having the formula (IVa) below:

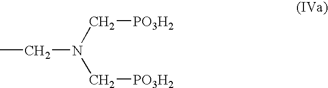

Especially preferred chelants according to formula (IV) for use herein are aminotri-(methylenephosphonic acid), ethylene-diamine-tetra-(methylenephosphonic acid) (EDTMP) and diethylene-triamine-penta-(methylenephosphonic acid) (DTPMP).

When used simultaneously with an oxidizing agent and a conditioning agents, chelants usually give good benefits above about 0.1%, preferably above about 0.25% by weight of the composition. For most chelants the best results were achieved at levels of above about 0.5% by weight. Levels above 5% can also be used but it would not be practical to use such high amounts for regulatory and safety reasons. Preferred range is therefore of from above about 0.5% to about 5%, more preferably of from above about 1% to about 4%, even more preferably of above about 2% to about 4%.

When used as a pretreatment (first composition) prior to the oxidizing step, chelants usually give good benefit at levels above 0.1%, preferably above 0.25%, more preferably above 0.5%, even more preferably above 1% by weight of said first composition used for the pretreatment (referred to in the claims and herein as “first composition”). For most chelants the best results were achieved at levels of about 2% by weight of the first composition.

When the conditioning agent is applied to hair as a post treatment after a first composition comprising a chelant and an oxidizing agent, said chelant is preferably present in the first composition at levels above 0.1%, preferably above 0.25%, more preferably above 0.5%, even more preferably above 1% by weight of said first composition. Levels above 5% can also be used but it would not be practical to use such high amounts for regulatory and safety reasons. Preferred range is therefore of from above about 2% to about 5%, more preferably of from above about 2% to about 4%.

Conditioning Agent

The compositions of the present invention comprise or are used in combination with a composition comprising a conditioning agent. Conditioning agents for use herein are selected from silicone materials, especially nonvolatile silicone and amino functionalised silicones, cationic surfactants, cationic polymers, alkoxylated amines and mixtures thereof.

The conditioning agent will generally be used at levels of from about 0.05% to about 20% by weight of the composition, preferably of from about 0.1% to about 15%, more preferably of from about 0.2% to about 10%, even more preferably of from about 0.2% to about 2%. The minimum level that is used in a particular composition should be effective to provide a conditioning benefit. The maximum level that can be used is not limited by theory, but rather by practicality. It is generally unnecessary and expensive to use levels in excess of about 10% and, depending on the type of agent (polymeric conditioners being most prone), such high levels can cause an undesirable weighting down of the hair.

When used as post-treatment immediately after the oxidizing composition has been applied and optionally rinsed off, the levels of conditioner in the post treatment composition are similar as those disclosed above, but by weight of the composition applied as a post-treatment.

Silicone Materials

Suitable silicone conditioning agents for use herein include nonvolatile and insoluble compound that intermix in the composition so as to be in the form of an emulsion, i.e., a separate, discontinuous phase of dispersed, insoluble droplets, usually suspended with a suspending agent. This dispersed silicone conditioning component will comprise a silicone fluid hair conditioning agent such as a silicone fluid and can also comprise other ingredients, such as a silicone resin to enhance silicone fluid deposition efficiency or, for example, enhance glossiness of the hair (especially when high refractive index (e.g. above about 1.46) silicone conditioning agents are used (e.g. highly phenylated silicones).

The silicone conditioning agent phase may comprise volatile silicone components. Typically, if volatile silicones are present, it will be incidental to their use as a solvent or carrier for commercially available forms of nonvolatile silicone materials ingredients, such as silicone gums and resins.

The silicone conditioning agent component for use herein will preferably have viscosity of from about 2.10⁻⁵ m²/s to about 2 m²/s (from about 20 to about 2,000,000 centistrokes at 25° C., more preferably from about 10⁻³ m²/s to about 1.8 m²/s (from about 1,000 to about 1,800,000 cSt), even more preferably from about 5.10⁻² m²/s to about 1.5 m²/s (from about 50,000 to about 1,500,000 cSt), most preferably from about 10⁻¹ m²/s to about 1.5 m²/s (from about 100,000 to about 1,500,000 cSt). The viscosity can be measured by means of a glass capillary viscometer as set forth in Dow Coming Corporate Test Method CTM0004, Jul. 20, 1970.

One type of silicone fluid that can be used herein is a silicone oil. The term “silicone oil” shall mean flowable silicone materials having a viscosity of less than 1 m²/s (1,000,000 centistokes) at 25° C. Generally, the viscosity of the fluid will be between about 5.10⁻⁶ m²/s and 1 m²/s (between about 5 and 1,000,000 centistokes) at 25° C., preferably between about 10⁻⁵ m²and 1.0 m²/s (between about 10 and about 100,000 cSt). Suitable silicone oils include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. More particularly silicone oils hereof include polyalkyl or polyaryl siloxanes having the general formula:

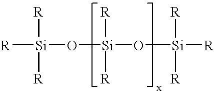

wherein R is aliphatic, preferably alkyl or alkenyl, or aryl, substituted or unsubstituted radical, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

The aliphatic or aryl groups substituted on the siloxane chain may have any structure as long as the resulting silicones remain fluid at room temperature, are hydrophobic, are neither irritating, toxic nor otherwise harmful when applied to the hair and/or skin, are compatible with the other components of the composition, are chemically stable under normal use and storage conditions, are insoluble in the composition, and are capable of being deposited on and, of conditioning, the hair and/or skin. The two R groups on the silicon atom of each monomeric silicone unit may represent the same group or different groups. Preferably, the two R groups represent the same group.

Preferred alkyl and alkenyl substituents are C₁-C₅ alkyls and alkenyls, more preferably from C₁-C₄, most preferably from C₁-C₂. The aliphatic portions of other alkyl-, alkenyl-, or alkynyl-containing groups (such as alkoxy, alkaryl, and alkamino) can be straight or branched chains and preferably have from one to five carbon atoms, more preferably from one to four carbon atoms, even more preferably from one to three carbon atoms, most preferably from one to two carbon atoms. As discussed above, the R substituents hereof can also contain amino functionalities, e.g. alkamino groups, which can be primary, secondary or tertiary amines or quaternary ammonium. These include mono-, di- and tri- alkylamino and alkoxyamino groups wherein the aliphatic portion chain length is preferably as described above. The R substituents can also be substituted with other groups, such as halogens (e.g. chloride, fluoride, and bromide), halogenated aliphatic or aryl groups, and hydroxy (e.g. hydroxy substituted aliphatic groups). Suitable halogenated R groups could include, for example, tri-halogenated (preferably fluoro) alkyl groups such as —R¹-C(F)₃, wherein R¹ is C₁-C₃ alkyl. Examples of such polysiloxanes include polymethyl-3,3,3trifluoropropylsiloxane.

The nonvolatile polyalkylsiloxane fluids that may be used include, for example, polydimethylsiloxanes. These siloxanes are available, for example, from the General Electric Company in their Viscasil R and SF 96 series, and from Dow Corning in their Dow Corning 200 series. Also suitable for use herein is Dow Coming DC 1664 (TN) 60,000 cstk polydimethyl siloxane with 300nm particle size which is preferably used in combination with a deposition aid. Polydimethyl silicone is also known as dimethicone oil. Other suitable R groups include methyl, methoxy, ethoxy, propoxy, and aryloxy. The three R groups on the end caps of the silicone may also represent the same or different groups. Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

Other suitable silicone conditioning agents are disclosed in WO9804237 p.22-p.29 incorporated herein by reference.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level must be sufficiently low to prevent solubility in water and the composition hereof. Alkylamino substituted silicones that can be used herein include those of the formula:

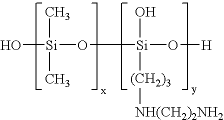

in which x and y are integers which depend on the molecular weight, the average molecular weight being approximately between 5,000 and 10,000. This polymer is also known as “amodimethicone”. Other aminosilicones suitable for use herein are available from the General Electric Company under the trade names SF1703, SM2115, SM2101, and SM2658.

Cationic Surfactants

Cationic surfactants useful in compositions of the present invention contain amino or quaternary ammonium hydrophilic moieties which are positively charged when dissolved in the aqueous composition of the present invention. Cationic surfactants containing quaternary ammonium moieties and silicone moieties can also be employed. Cationic surfactants among those useful herein are disclosed in the following documents, all incorporated by reference herein: M.C. Publishing Co., McCutcheon's Detergents & Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin, et al., issued Dec. 30, 1975; U.S. Pat. No. 3,959,461, Bailey, et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

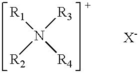

wherein R₁-R₄ are independently an aliphatic group of from about 1 to about 22 carbon atoms, aryl, or an alkoxy, polyoxyalkylene, alkylamido, hydroxyalkyl, or alkylaryl group having from about 1 to about 22 carbon atoms; and X is an anion selected from halogen (especially chlorine), acetate, phosphate, nitrate and alkylsulfate (preferably C₁-C₃ alkyl) radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated.

Quaternary ammonium salts include dialkyldimethyl-ammonium chlorides, wherein the alkyl groups have from about 12 to about 22 carbon atoms and are derived from long-chain fatty acids, such as hydrogenated tallow fatty acid (tallow fatty acids yield quaternary compounds wherein R₁ and R₂ have predominately from 16 to 18 carbon atoms). Other cationic surfactants include those wherein at least one of the R₁-R₄ radicals contains one or more hydrophilic moieties selected from alkoxy (preferably C₁-C₃ alkoxy), polyoxyalkylene (Preferably C₁-C₃ polyoxyalkylene), alkylamido, hydroxyalkyl, alkylester, and combinations thereof. Optionally, the cationic conditioning surfactant contains from 2 to about 10 nonionic hydrophile moieties in the R₁-R₄ groups. For purposes herein, each hydrophilic amido, alkoxy, hydroxyalkyl, alkylester, alkylamido or other unit is considered to be a distinct nonionic hydrophile moiety.

Other cationic surfactants useful herein are disclosed in WO9632919 incorporated herein by reference.

Cationic Polymers

Suitable cationic polymers are disclosed in WO9632919 p.17-21, incorporated herein by reference. Examples of cationic polysaccharide polymer materials suitable for use herein include those of the formula:

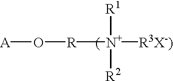

wherein:

A is an anhydroglucose residual group, such as a starch or cellulose anhydroglucose residual,

R is an alkylene oxyalkylene, polyoxyalkylene, or hydroxyalkylene group, or combination thereof,

R¹, R², and R³ independently are alkyl, aryl, alkyl, aryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less, and

X is an anionic counterion, as previously described.

Cationic cellulose is available from Amerchol Corp. (Edison, N.J., USA) in their Polymer JR^{TN}, LR^{TN}, KG^{TN} and LK^{TN} series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200 (or Quatrisoft).

Alkoxylated Amines

Alkoxylated amines are conditioning agents conforming to the formula:

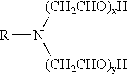

wherein R is an alkyl, preferably having from 8 to 30 carbon atoms, and x and y are integers, preferably wherein x+y is in the range of from 1 to 100, more preferably 50, event more preferably of between 1 to 20.

Oxidizing Agents

The compositions according to the present invention comprise or are used in combination with a composition that comprises at least one oxidizing agent. Preferred oxidizing agents for use herein are water-soluble peroxygen oxidizing agents. “Water-soluble” as defined herein means that in standard condition at least 0.1 g, preferably 1 g, more preferably 10 g of said oxidizing agent can be dissolved in 1 liter of deionized water. The oxidizing agents are valuable for the initial solubilisation and decolorisation of the melanin (bleaching) and accelerate the polymerization of the oxidative dye precursors (oxidative dyeing) in the hair shaft.

Preferred water-soluble oxidizing agents are inorganic peroxygen materials capable of yielding hydrogen peroxide in an aqueous solution. Water-soluble peroxygen oxidizing agents are well known in the art and include hydrogen peroxide, inorganic alkali metal peroxides such as sodium periodate and sodium peroxide and organic peroxides such as urea peroxide, melamine peroxide, and inorganic perhydrate salt bleaching compounds, such as the alkali metal salts of perborates, percarbonates, perphosphates, persilicates, persulphates and the like. These inorganic perhydrate salts may be incorporated as monohydrates, tetrahydrates etc. Mixtures of two or more such oxidizing agents can be used if desired. Preferred for use in the compositions according to the present invention is hydrogen peroxide.

In conventional dyeing and bleaching compositions, levels of peroxygen oxidizing agent are usually of from about 0.1% to about 7% by weight. Higher levels, whilst giving good results in term of efficacy were until now not practical because of increased hair damage. The oxidative damage protection provided by the present invention makes it now possible to use oxidizing agent such as hydrogen peroxide in level up to 40% in the oxidizing composition. However, for safety reasons, level above 12% should be carefully investigated before being used on human. Preferably, the level of the oxidizing agent in the oxidizing composition is of from about 0.5% to about 20% by weight, more preferably of from about 1% to about 15%. The compositions according to the present invention provide excellent gray coverage, vibrant colors and acceptable damage at level above about 7% (typically about 12%).

Additional Components

Moreover, it is also intended that the compositions of the present invention may be complex compositions, which in addition to the chelant and oxidizing agent comprise other components that may or may not be active ingredients. This includes, but is not limited to, buffering agents, hair dyeing agents such as oxidative dye precursors, non-oxidative dyes, thickeners, solvents, enzymes, anionic, non ionic, amphoteric and cationic surfactants, carriers, antioxidants, stabilizers, perming actives, perfume, hair swelling agents and/or polymers. Some of these additional components are detailed hereafter.

Finally, the compositions according to the present invention can be provided in any usual form, such as for example an aqueous composition, a powder, a gel or an oil-in-water emulsion. Preferred media for the compositions according to the present invention are thickened aqueous solutions comprising a salt-tolerant thickener or oil-in-water emulsions.

pH Buffering Agents

The compositions according to the present invention preferably further comprise a pH buffering agent. The pH of the composition is preferably of from about 8 to about 12, more preferably of from about 9 to about 11, even more preferably of from about 9.5 to about 10.5. Suitable buffering agents are well known in the art and include for example an ammonia/ammonium acetate mixture and monoethanolamine (MEA).

Oxidative Hair Dye Precursors

These compounds are well known in the art, and include aromatic diamines, aminophenols and their derivatives (a representative but not exhaustive list of oxidation dye precursor can be found in Sagarin, “Cosmetic Science and Technology”, “Interscience, Special Edn. Vol. 2 pages 308 to 310). Precursors can be used with couplers. Couplers are generally colorless molecules that can form colors in the presence of activated precursors.

The choice of precursors and couplers will be determined by the color, shade and intensity of coloration that is desired. The precursors and couplers can be used herein, singly or in combination, to provide dyes having a variety of shades ranging from ash blonde to black.

Hair dye compositions will generally comprise from about 0.001% to about 10%, preferably from about 0.1% to about 2%, of oxidative dye precursors and couplers.

Thickeners

The composition of the present invention may optionally further comprise at least about 0.1% of thickeners. Thickeners are preferably comprised in amount sufficient to provide the composition with a viscosity of from about 1 Pa.s to 10 Pa.s (1,000 to 10,000 cP) at 26° C. in order to provide a composition that can be readily applied to the hair without dripping.

Preferred for use herein are salt tolerant thickeners. Salt-tolerant thickeners are functionally defined herein as compounds that increases the viscosity of an aqueous composition consisting of 3.8% DTPMP (tetrasodium salt) and 1.95% NH₃ to at least 1 Pa.s (1,000 cP) when incorporated at levels of 2% by weight as measured at 26.7° C. The viscosity can be measured with a Brookfield viscometer DVII, using S41 spindles for samples under 10 Pa.s (10,000 cP) and spindle S52 for samples over 10 Pa.s (10,000 cP) (available from Brookfield), with a speed rating of 1 revolution per minute and samples sizes of 2 ml (for S41 spindle) or 0.5 ml (for S52 spindle).

A non exclusive list of suitable salt tolerant thickeners for use herein include xanthan, guar, hydroxypropyl guar, scleroglucan, methyl cellulose, ethyl cellulose (commercially available as Aquacote®), hydroxyethyl cellulose (Natrosol®), carboxymethyl cellulose, hydroxypropylmethyl cellulose, microcrystalline cellulose, hydroxybutylmethyl cellulose, hydroxypropyl cellulose (Klucel®), hydroxyethyl ethyl cellulose, cetyl hydroxyethyl cellulose (Natrosol® Plus 330), N-vinylpyrollidone (Povidone®), Acrylates/Ceteth-20 Itaconate Copolymer (Structure® 3001), hydroxypropyl starch phosphate (Structure® ZEA), polyethoxylated urethanes or polycarbamyl polyglycol ester (e.g. PEG-150/Decyl/SMDI copolymer=Aculyn® 44, PEG-150/Stearyl/SMDI copolymer=Aculyn 46®), trihydroxystearin (Thixcin®) acrylates copolymer (e.g. Aculyn® 33) or hydrophobically modified acrylate copolymers (e.g. Acrylates/Steareth-20 Methacrylate Copolymer=Aculyn® 22).

Fatty alcohols have thickening properties and can be used in the compositions present invention. Fatty alcohols are however not salt-tolerant thickeners according to the above definition. A mixture of 2% cetyl and stearyl alcohol has for example a viscosity of less than about 0.7 Pa.s (700 cP) as measured at 26° C. with a Brookfield viscometer in the conditions disclosed hereabove.

### EXAMPLES

The following examples illustrate oxidative dye compositions according to the present invention. It is understood that the examples and embodiments described herein are for illustrative purposes only and that various modifications or changes in light thereof will be suggested to one skilled in the art without departing from the scope of the present invention.

Examples of formulation: emulsion

| | 1 | 2 | 3 | 4 | 5 | 6 | 7 | 8 | 9 | 10 |
|---|---|---|---|---|---|---|---|---|---|---|
| Sodium sulphite | 0.1 | 0.1 | 0.1 | 0.1 | 0.1 | 0.1 | 0.1 | 0.1 | 0.1 | 0.1 |
| Ascorbic Acid | 0.1 | 0.1 | 0.1 | 0.1 | 0.1 | 0.1 | 0.1 | 0.1 | 0.1 | 0.1 |
| Ammonium Acetate | 1.5 | 1.5 | 1.5 | 1.5 | 1.5 | 1.5 | 1.5 | 1.5 | 1.5 | 1.5 |
| Ammonia | 6.0 | 6.0 | 6.0 | 6.0 | 6.0 | 6.0 | 6.0 | 6.0 | 6.0 | 6.0 |
| (30% active) | | | | | | | | | | |
| Ceteareth 25 | 1.0 | 1.0 | 1.0 | 1.0 | 1.0 | 1.0 | 1.0 | 1.0 | 1.0 | 1.0 |
| Cetyl Alcohol | 1.6 | 1.6 | 1.6 | 1.6 | 1.6 | 1.6 | 1.6 | 1.6 | 1.6 | 1.6 |
| Stearyl Alcohol | 3.3 | 3.3 | 3.3 | 3.3 | 3.3 | 3.3 | 3.3 | 3.3 | 3.3 | 3.3 |
| Sodium Benzoate | 0.1 | 0.1 | 0.1 | 0.1 | 0.1 | 0.1 | 0.1 | 0.1 | 0.1 | 0.1 |
| Phenoxyethanol | 0.1 | 0.1 | 0.1 | 0.1 | 0.1 | 0.1 | 0.1 | 0.1 | 0.1 | 0.1 |
| HEDP | 1.5 | — | — | — | 2.5 | 1.0 | — | 1.0 | 0.5 | 5.0 |
| (disodium salt of) | | | | | | | | | | |
| EDTMP | — | 0.6 | | 1.0 | — | 0.5 | 0.5 | — | 1.5 | — |
| (tetrasodium salt of) | | | | | | | | | | |
| DTPMP | — | — | 1 | 1.0 | 0.5 | 1.0 | 1.0 | 1.0 | 1.0 | — |
| (tetrasodium salt of) | | | | | | | | | | |
| Para-phenylene diamine | 0.8 | 0.5 | 0.6 | 0.5 | 0.8 | 0.8 | 0.5 | 0.6 | 0.5 | 0.8 |
| Para-aminophenol | 0.2 | 0.3 | 0.2 | 0.1 | 0.2 | 0.2 | 0.3 | 0.2 | 0.1 | 0.2 |
| Meta-aminophenol | 1.0 | 0.5 | 1.0 | 0.6 | 1.0 | 1.0 | 0.5 | 1.0 | 0.6 | 1.0 |
| Resorcinol | 1.6 | 1.2 | 1.6 | 0.8 | 1.6 | 1.6 | 1.2 | 1.6 | 0.8 | 1.6 |
| Hydrogen Peroxide | 8.6 | 8.6 | 8.6 | 13 | 17 | 17 | 17 | 34 | 34 | 34 |
| (35% active) | | | | | | | | | | |
| Trimethylsilylamodi- | 0.5 | 0.5 | 1.5 | 2.0 | 2.0 | 2.0 | 2.0 | 2.0 | 2.0 | 2.0 |
| methicone (SF1708) | | | | | | | | | | |
| Polyquaternium 10 | 0.2 | 0.2 | — | 0.2 | 0.2 | 0.2 | 0.2 | — | — | — |
| (Polymer JR30M) | | | | | | | | | | |
| Xanthan gum | 0.5 | 0.5 | — | 1.0 | 0.5 | 0.5 | 0.5 | 0.5 | 0.5 | 1.0 |
| Cetyl hydroxyethyl | — | — | 0.8 | 1.0 | 0.8 | 0.5 | 0.5 | 0.5 | 0.5 | 1.0 |
| Cellulose | | | | | | | | | | |
| (Natrosol 330CS Plus) | | | | | | | | | | |
| pH adjust to pH 10 | qs | qs | qs | qs | qs | qs | qs | qs | qs | qs |
| Water | qs | qs | qs | qs | qs | qs | qs | qs | qs | qs |

### Example of Formulation: Thickened Aqueous Solution

Example of formulation: thickened aqueous solution

| | 1 | 2 | 3 | 4 | 5 | 6 | 7 | 8 | 9 | 10 |
|---|---|---|---|---|---|---|---|---|---|---|
| Sodium sulphite | 0.1 | 0.1 | 0.1 | 0.1 | 0.1 | 0.1 | 0.1 | 0.1 | 0.1 | 0.1 |
| Ascorbic Acid | 0.1 | 0.1 | 0.1 | 0.1 | 0.1 | 0.1 | 0.1 | 0.1 | 0.1 | 0.1 |
| Citric Acid | 1.0 | 1.0 | 1.0 | 1.0 | 1.0 | 1.0 | 1.0 | 1.0 | 1.0 | 1.0 |
| Ammonia | 6.0 | 6.0 | 6.0 | 6.0 | 6.0 | 6.0 | 6.0 | 6.0 | 6.0 | 6.0 |
| (30% active) | | | | | | | | | | |
| Acrylates Copolymer | 2.4 | 2.4 | 2.4 | 2.4 | 2.4 | 2.4 | 2.4 | 2.4 | 2.4 | 2.4 |
| (Aculyn 33A) | | | | | | | | | | |
| Oleth 10 | 1.0 | 1.0 | 1.0 | 1.0 | 1.0 | 1.0 | 1.0 | 1.0 | 1.0 | 1.0 |
| Oleth 2 | 0.8 | 0.8 | 0.8 | 0.8 | 0.8 | 0.8 | 0.8 | 0.8 | 0.8 | 0.8 |
| Oleic Acid | 0.9 | 0.9 | 0.9 | 0.9 | 0.9 | 0.9 | 0.9 | 0.9 | 0.9 | 0.9 |
| Cocamide DEA | 3.0 | 3.0 | 3.0 | 3.0 | 3.0 | 3.0 | 3.0 | 3.0 | 3.0 | 3.0 |
| HEDP | 1.5 | — | — | — | 2.5 | 1.0 | — | 1.0 | 0.5 | 5.0 |
| (disodium salt of) | | | | | | | | | | |
| EDTMP | — | 0.6 | | 1.0 | — | 0.5 | 0.5 | — | 1.5 | — |
| (tetrasodium salt of) | | | | | | | | | | |
| DTPMP | — | — | 1 | 1.0 | 0.5 | 1.0 | 1.0 | 1.0 | 1.0 | |
| (tetrasodium salt of) | | | | | | | | | | |
| Para-phenylene diamine | 0.8 | 0.5 | 0.6 | 0.5 | 0.8 | 0.8 | 0.5 | 0.6 | 0.5 | 0.8 |
| Para-aminophenol | 0.2 | 0.3 | 0.2 | 0.1 | 0.2 | 0.2 | 0.3 | 0.2 | 0.1 | 0.2 |
| Meta-aminophenol | 1.0 | 0.5 | 1.0 | 0.6 | 1.0 | 1.0 | 0.5 | 1.0 | 0.6 | 1.0 |
| Resorcinol | 1.6 | 1.2 | 1.6 | 0.8 | 1.6 | 1.6 | 1.2 | 1.6 | 0.8 | 1.6 |
| Hydrogen Peroxide | 8.6 | 8.6 | 8.6 | 13 | 17 | 17 | 17 | 34 | 34 | 34 |
| (35% active) | | | | | | | | | | |
| Behentrimonium | 0.5 | 0.5 | 1.5 | 2.0 | 2.0 | 2.0 | 2.0 | 2.0 | 2.0 | 2.0 |
| Chloride | | | | | | | | | | |
| Dicetyldimonium | 0.2 | 0.2 | 0.7 | 0.2 | 0.2 | 0.2 | 0.2 | — | — | — |
| Chloride | | | | | | | | | | |
| Acrylates Steareth-20 | 0.5 | 0.5 | 0.0 | 1.0 | 0.5 | 0.5 | 0.5 | 0.5 | 0.5 | 1.0 |
| Methacrylate Copolymer | | | | | | | | | | |
| (Aculyn 22) | | | | | | | | | | |
| Propylene Glycol | 8.2 | 8.2 | 8.2 | 8.2 | 8.2 | 8.2 | 8.2 | 8.2 | 8.2 | 8.2 |
| Ethoxy Diglycol | 4.2 | 4.2 | 4.2 | 4.2 | 4.2 | 4.2 | 4.2 | 4.2 | 4.2 | 4.2 |
| pH adjust to pH 10 | qs | qs | qs | qs | qs | qs | qs | qs | qs | qs |
| Water | qs | qs | qs | qs | qs | qs | qs | qs | qs | qs |

The above compositions are useful for dyeing hair with reduced damage and improved conditioner deposition. Similar compositions not including oxidative dye precursors and couplers (in the above examples para-aminophenol, meta-aminophenol and resorcinol) can be used for bleaching (lightening) hair.

Oxidative hair dye compositions are usually sold in kits comprising, in separate containers, a dye component (also called “dye cream” for emulsion or “dye liquid” for solution) comprising the oxidative dye precursors (and usually the Hair Swelling Agent) and a hydrogen peroxide component (also called “hydrogen peroxide cream” for emulsion or “hydrogen peroxide liquid” for solution) comprising the oxidizing agent (usually hydrogen peroxide). The consumer mixes the dye component and hydrogen peroxide component immediately before use. The examples of the tables above illustrate the resulting mixtures.

Similarly, bleaching compositions are usually sold as a kit comprising two or three separate containers. The first contains the hair-swelling agent (e.g. ammonia), the second contains the oxidizing agent and the third (optional) contains a second oxidizing agent (e.g. alkali or ammonium salt of persulphates, percarbonate, perborate). The bleaching compositions are obtained by mixing the above-mentioned compositions immediately before use.

These kits are well known in the art and the composition in each container can be manufactured utilizing any one of the standard approaches, these include:

Oil in water process

Phase Inversion process

One-pot process

The chelants are usually added to a proportion of the water at the start of the making process at ambient temperature, and allowed to dissolve. The fatty components are then added and the mixture is processed as is normal for the above outlined procedures. For example, in a 1 pot process the polymers and chelants would be predissolved in water, the fatty materials added and then the whole heated to about 70-80° C.

A controlled cooling and optional shearing process to form the final structured product in the case of an emulsion would then follow. Addition of the ammonia and pH trimming complete the making process of the dye cream.

In the case of a liquid solution comprising acrylate polymers, these would be formulated into the hydrogen peroxide component. The glycol solvents and fatty components are formulated into the dye component. A structured product is formed when the dye and hydrogen peroxide components are mixed together prior to use of the composition, through deprotonation of the polymer acrylic acid groups yielding a polymeric micro-gel. Further details on the manufacture of these two-part aqueous composition for coloring hair, which forms a gel on mixing of the two parts can be found in U.S. Pat. No. 5,376,146, Casperson et al. and U.S. Pat. No. 5,393,305, Cohen et al.

The composition of the present invention can also be formulated as 2-part aqueous compositions comprising polyetherpolyurethane as thickening agent (such as Aculyn® 46) as described in U.S. Pat. No. 6,156,076, Casperson et al. and U.S. Pat. No. 6,106,578, Jones.

When the compositions of different containers are mixed before use and the resulting mixture comprises the chelants and conditioning agents claimed, there is no preference on how the chelants and conditioning agents are distributed in these containers. Obviously chelants that can be altered by hydrogen peroxide (or any oxidizing agent used) such as secondary amine chelants should however be formulated in the dye component. The hydrogen peroxide component should however preferably comprise at least about 0.1% of a stable chelant to stabilize hydrogen peroxide. This stabilizer (which is not necessary a phosphonate chelant) is required to prevent the hydrogen peroxide from decomposing too rapidly. For example EDTA can be used in the hydrogen peroxide component as stabilizer.

### METHODS OF USE

It is understood that the examples of methods of use and embodiments described herein are for illustrative purposes only and that various modifications or changes in light thereof will be suggested to one skilled in the art without departing from the scope of the present invention.

Without Pretreatment

The chelants according to the present invention are preferably formulated directly in the oxidizing compositions applied on hair (e.g. oxidative dye composition or bleach).

Oxidative Dye

Oxidative dye compositions are usually sold as a kit comprising at least two separate containers: one contains the oxidative dye precursors with the hair-swelling agent (e.g. ammonia) in a suitable carrier (e.g. dye cream or liquid) and the other contains the oxidizing agent in a suitable carrier (e.g. hydrogen peroxide cream or liquid). The consumer prepares the oxidative dye composition immediately before use by mixing both compositions and applies it on hair. After working the mixture a few minutes (to insure uniform application to all of the hair), the oxidative dye composition is allowed to remain on the hair for an amount sufficient for the dyeing to take place (usually about 30 minutes). The consumer then rinses his/her hair thoroughly with tap water and allows it to dry. It is observed that the hair has changed from its original color to the desired color.

The conditioning agent can also be packaged partly or in totality in a third container. In this case, all three compositions can be mixed immediately before use and applied together, or the content of the third container can be applied (with an optional rinse step) as post-treatment immediately after the oxidative dye composition resulting from the mixture of the other containers.

Bleaching Compositions Bleaching compositions are usually sold as a kit comprising two or three separate containers. The first contains the hair-swelling agent (e.g. ammonia), the second contains the oxidizing agent and the third (optional) contains a second oxidizing agent (e.g. alkali or ammonium salt of persulphates, percarbonate, perborate). The consumer prepares the bleaching compositions immediately before use by mixing all compositions and applies the mixture on hair (as for the oxidative dye composition) for an amount of time sufficient for the bleaching to take place (usually about 30mn).

The conditioning agent can also be packaged partly or in totality in an additional third or fourth container. In this case, all compositions can be mixed together immediately before use or the content of the additional container can be applied (with an optional preliminary rinse step) as a post-treatment immediately after the bleaching composition resulting from the mixture of the other containers.

With Pretreatment

The chelants and conditioners can also be applied to hair as a pre-treatment. The pretreatment composition (“first composition”) can be applied immediately before the oxidizing composition (“second composition”) or after a longer period of time.

Pretreatment Immediately Followed by an Oxidizing Composition

In the case of a pretreatment applied on hair and immediately followed by the oxidizing composition, said pretreatment composition can be rinsed off hair before the application of the oxidizing composition, but will be preferably kept on the hair during the application of the oxidizing compositions, the resulting mixture being rinsed off following the oxidizing step. Kits comprising one container for the first composition (pre-treat) and one, two or more containers for the second composition (oxidizing composition) can be advantageously used for this method. Two containers or more can be required for the second composition in case this second composition is prepared immediately before use by mixing the content of two containers or more (e.g. oxidative hair dye composition or bleaching composition). The kit can also comprise an additional container for a composition comprising a conditioning agent that is applied independently from the second composition in a third step, optionally following a rinsing step.

When the pretreatment comprises a chelant and a conditioning agent, the conditioning agent is selected from silicone materials, especially nonvolatile silicone and amino functionalised silicones, cationic polymers, alkoxylated amines and mixtures thereof.

Color Care

The pretreatment can also take place as a “color care” treatment anytime between two oxidative treatments but not immediately prior to one. The 2 oxidative treatments are preferably separated by at least one day, more preferably at least one week. Oxidative hair dye treatments are generally repeated about once a month and obviously, hair will be usually rinsed with water immediately after each oxidative treatment. The “color care” treatment can be repeated as many times as practical between these 2 oxidative treatments, which can be once, twice or more.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A composition suitable for treating hair comprising:
a) an oxidizing agent;
b) a conditioning agent selected from the group consisting of silicone materials, cationic surfactants, cationic polymers, alkoxylated amines and mixtures thereof;
c) a chelant selected from the group consisting of phosphonic acid type chelants, and salts thereof, derivatives thereof and mixtures thereof.

2. A composition according to claim 1, wherein said chelant is selected from the group consisting of chelants having the formula (I) to (III) below:
i) formula (I): 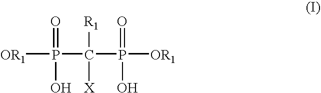
wherein X is —OH or —NH₂; R₁ is an aryl radical or an aliphatic radical having 1 to 5 carbon atoms or a group having the formula (Ia) below: 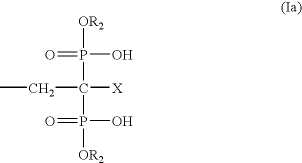
wherein each R₂ is independently selected from H or an alkyl radical having from 1 to 5 carbon atoms;
ii) formula (II): 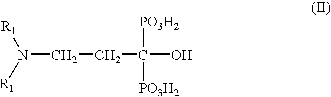
wherein each R₁ is independently selected from H or C₁-C₃ alkyl; and iii) formula (III): 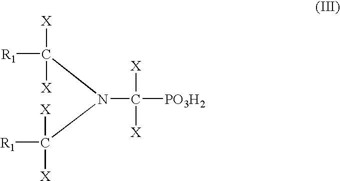
wherein each X is independently selected from hydrogen or alkyl radicals; and
each R₁ is independently selected from —O₃H₂ or a group having the formula (IIa) below: 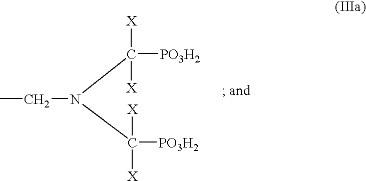
salts thereof, derivatives thereof and mixtures thereof.

3. A composition according to claim 2, wherein said chelant is selected from the group consisting of aminotri-(1-ethylphosphonic acid), ethylene-diaminetetra-(1-ethylphosphonic acid), aminotri-(1-propylphosphonic acid), aminotri-(isopropyl-phosphonic acid), and salts thereof, derivatives thereof and mixtures thereof.

4. A composition according to claim 1, wherein said chelant is selected from the group consisting of chelants having the formula (IV) below: 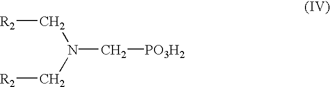
wherein each R₂ is independently selected from —PO₃H₂ or a group having the formula (IVa) below: 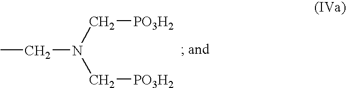
salts thereof, derivatives thereof and mixtures thereof.

5. A composition according to claim 4, wherein said chelant is selected from the group consisting of aminotri-(methylenephosphonic acid), ethylene-diaminotetra-(methylenephosphonic acid) (EDTMP), diethylene-triamine-penta-(methylenephosphonic acid) (DTPMP), and salts thereof, derivatives thereof, and mixtures thereof.

6. A composition according to claim 1, wherein the pH is between about 8 and about 12.

7. A composition according to claim 1 wherein said composition is in the form of an oil-in-water emulsion.

8. A composition according to claim 1 wherein said composition is in the form of a thickened aqueous solution.

9. A composition according to claim 1, further comprising a salt-tolerant thickener selected from the group consisting of xanthan, guar, hydroxypropyl guar, scleroglucan, methyl cellulose, ethyl cellulose, hydroxyethyl cellulose, carboxymethyl cellulose, hydroxypropylmethyl cellulose, microcrystalline cellulose, hydroxybutylmethyl cellulose, hydroxypropyl cellulose, hydroxyethyl ethyl cellulose, cetyl hydroxyethyl cellulose, N-vinylpyrollidone, Acrylates/Ceteth-20 Itaconate Copolymer, hydroxypropyl starch phosphate, polyethoxylated urethanes or polycarbamyl polyglycol ester, trihydroxystearin, acrylates copolymer or hydrophobically modified acrylate copolymers and mixtures thereof.

10. A composition according to claim 1, further comprising at least one oxidative hair dye precursor.

11. A method of treating hair, said method comprising the steps of:
i) contacting hair with a first composition comprising:
a) a conditioning agent selected from the group consisting of silicone materials, cationic polymers, alkoxylated amines and mixtures thereof;
b) a chelant selected from the group consisting of phosphonic acid type chelants, salts thereof, derivatives thereof and mixtures thereof; and
ii) contacting hair immediately after step i) with a second composition comprising an oxidizing agent.

12. A method according to claim 11, wherein said second composition further comprises an oxidative hair dye precursor.

13. A method of treating hair, said method comprising the steps of:
i) contacting hair with a first composition comprising an oxidizing agent;
ii) contacting hair with a second composition comprising a conditioning agent and a chelant selected from the group consisting of phosphonic type chelants, and salts thereof, derivatives thereof and mixtures thereof; and
iii) contacting hair with a third composition comprising a second oxidizing agent;
wherein steps i) and ii) are separated by at least 1 day and that step ii) does not take place immediately before step iii).

14. A method of treating hair, said method comprising the steps of:
i) contacting hair with a first composition comprising a chelant selected from the group consisting of phosphonic acid type chelants, and salts thereof, derivatives thereof and mixtures thereof;
ii) contacting hair with a second composition comprising an oxidizing agent immediately after step i);
iii) contacting hair with a third composition comprising a conditioning agent immediately after step iii).

15. A kit for dyeing hair comprising a first and a second compositions packaged in different containers, wherein said first composition comprises an oxidizing agent and said second composition comprises an oxidative dye precursor, wherein the resulting mixture of said first and second compositions is a composition according to claim 10.
